SEQUENCE LISTING (16 sequences)

SEQ ID NO: 1 - protein, 148 aa, human
MRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARK

SEQ ID NO: 2 - protein, 147 aa, human
IRVGTNYQAVIPECKPESPARYSNKELKGMLVWSPNHCVSDAKLDKYIAMAKEKHGYNIEQALGMLLWHKHDVEKSLADLANFTPFPDEWTVEDKVLFEQAFGFHGKCFQRIQQMLPDKLIPSLVKYYYSWKKTRSRTSVMDRQARR

SEQ ID NO: 3 - protein, 146 aa, human
MRVGAEYQARIPEFDPGATKYTDKDNGGMLVWSPYHSIPDAKLDEYIAIAKEKHGYNVEQALGMLFWHKHNIEKSLADLPNFTPFPDEWTVEDKVLFEQAFSFHGKSFHRIQQMLPDKTIASLVKYYYSWKKTRSRTSLMDRQARK

SEQ ID NO: 4 - protein, 180 aa, human
IRVGNRYQADITDLLKEGEEDGRDQSRLETQVWEAHNPLTDKQIDQFLVVARSVGTFARALDCSSSVRQPSLHMSAAAASRDITLFHAMDTLHKNIYDISKAISALVPQGGPVLCRDEMEEWSASEANLFEEALEKYGKDFTDIQQDFLPWKSLTSIIEYYYMWKTTDRYVQQKRLKAAE

SEQ ID NO: 5 - protein, 180 aa, human
IRVGCKYQAEIPDRLVEGESDNRNQQKMEMKVWDPDNPLTDRQIDQFLVVARAVGTFARALDCSSSIRQPSLHMSAAAASRDITLFHAMDTLQRNGYDLAKAMSTLVPQGGPVLCRDEMEEWSASEAMLFEEALEKYGKDFNDIRQDFLPWKSLASIVQFYYMWKTTDRYIQQKRLKAAE

SEQ ID NO: 6 - protein, 180 aa, human
IRVGPRYQADIPEMLLEGESDEREQSKLEVKVWDPNSPLTDRQIDQFLVVARAVGTFARALDCSSSVRQPSLHMSAAAASRDITLFHAMDTLYRHSYDLSSAISVLVPLGGPVLCRDEMEEWSASEASLFEEALEKYGKDFNDIRQDFLPWKSLTSIIEYYYMWKTTDRYVQQKRLKAAE

SEQ ID NO: 7 - protein, 168 aa, human
IMVGSMFQAEIPVGICRYKENEKVYENDDQLLWDPEYLPEDKVIIFLKDASRRTGDEKGVEAIPEGSHIKDNEQALYELVKCNFDTEEALRRLRFNVKAAREELSVWTEEECRNFEQGLKAYGKDFHLIQANKVRTRSVGECVAFYYMWKKSERYDFFAQQTRFGKKK

SEQ ID NO: 8 - protein, 167 aa, human
IMVGPQFQADLSNLHLNRHCEKIYENEDQLLWDPSVLPEREVEEFLYRAVKRRWHEMAGPQLPEGEAVKDSEQALYELVKCNFNVEEALRRLRFNVKVIRDGLCAWSEEECRNFEHGFRVHGKNFHLIQANKVRTRSVGECVEYYYLWKKSERYDYFAQQTRLGRRK

SEQ ID NO: 9 - protein, 167 aa, human
IMIGLQYQAEIPPYLGEYDGNEKVYENEDQLLWCPDVVLESKVKEYLVETSLRTGSEKIMDRISAGTHTRDNEQALYELLKCNHNIKEAIERYCCNGKASQGMTAWTEEECRSFEHALMLFGKDFHLIQKNKVRTRTVAECVAFYYMWKKSERYDYFAQQTRFGKKR

SEQ ID NO: 10 - protein, 171 aa, human
IRVGPSHQAKLPDLQPFPSPDGDTVTQHEELVWMPGVNDCDLLMYLRAARSMAAFAGMCDGGSTEDGCVAASRDDTTLNALNTLHESGYDAGKALQRLVKKPVPKLIEKCWTEDEVKRFVKGLRQYGKNFFRIRKELLPNKETGELITFYYYWKKTPEAASSRAHRRHRRQ

SEQ ID NO: 11 - protein, 167 aa, human
INIGLRFQAEIPELQDISALAQDTHKATLVWKPWPELENHDLQQRVENLLNLCCSSALPGGGTNSEFALHSLFEAKGDVMVALEMLLLRKPVRLKCHPLANYHYAGSDKWTSLERKLFNKALATYSKDFIFVQKMVKSKTVAQCVEYYYTWKKIMRLGRKHRTRLAE

SEQ ID NO: 12 - protein, 168 aa, human
INIGSRFQAEIPELQERSLAGTDEHVASLVWKPWGDMMISSETQDRVTELCNVACSSVMPGGGTNLELALHCLHEAQGNVQVALETLLLRGPHKPRTHLLADYRYTGSDVWTPIEKRLFKKAFYAHKKDFYLIHKMIQTKTVAQCVEYYYIWKKMIKFDCGRAPGLEK

SEQ ID NO: 13 - protein, 822 aa, Artificial Sequence
  GAL4 DBD-MTA2
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFGSGSMAANMYRVGDYVYFENSSSNPYLVRRIEELNKTANGNVEAKVVCLFRRRDISSSLNSLADSNAREFEEESKQPGVSEQQRHQLKHRELFLSRQFESLPATHIRGKCSVTLLNETDILSQYLEKEDCFFYSLVFDPVQKTLLADQGEIRVGCKYQAEIPDRLVEGESDNRNQQKMEMKVWDPDNPLTDRQIDQFLVVARAVGTFARALDCSSSIRQPSLHMSAAAASRDITLFHAMDTLQRNGYDLAKAMSTLVPQGGPVLCRDEMEEWSASEAMLFEEALEKYGKDFNDIRQDFLPWKSLASIVQFYYMWKTTDRYIQQKRLKAAEADSKLKQVYIPTYTKPNPNQIISVGSKPGMNGAGFQKGLTCESCHTTQSAQWYAWGPPNMQCRLCASCWIYWKKYGGLKTPTQLEGATRGTTEPHSRGHLSRPEAQSLSPYTTSANRAKLLAKNRQTFLLQTTKLTRLARRMCRDLLQPRRAARRPYAPINANAIKAECSIRLPKAAKTPLKIHPLVRLPLATIVKDLVAQAPLKPKTPRGTKTPINRNQLSQNRGLGGIMVKRAYETMAGAGVPFSANGRPLASGIRSSSQPAAKRQKLNPADAPNPVVFVATKDTRALRKALTHLEMRRAARRPNLPLKVKPTLIAVRPPVPLPAPSHPASTNEPIVLED

SEQ ID NO: 14 - protein, 334 aa, Artificial Sequence
  GAL4 DBD-MTA2-X
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFGSGSIRVGCKYQAEIPDRLVEGESDNRNQQKMEMKVWDPDNPLTDRQIDQFLVVARAVGTFARALDCSSSIRQPSLHMSAAAASRDITLFHAMDTLQRNGYDLAKAMSTLVPQGGPVLCRDEMEEWSASEAMLFEEALEKYGKDFNDIRQDFLPWKSLASIVQFYYMWKTTDRYIQQKRLKAAE

SEQ ID NO: 15 - protein, 636 aa, Artificial Sequence [sequence omitted]

SEQ ID NO: 16 - protein, 302 aa, Artificial Sequence
  GAL4 DBD-CoREST-X
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFGSGSMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARK

(19) United States
(12) Patent Application Publication
(10) Pub. No.: US 2009/0298046 A1
(43) Pub. Date: Dec. 3, 2009

(54) Assays for Histone Deacetylase 1/2 Selective Inhibitors

(75) Inventors: Armin Lahm, Rome (IT); Paola Gallinari, Rome (IT)

Correspondence Address: MERCK AND CO., INC, P O BOX 2000, RAHWAY, NJ, 07065-0907, US

(73) Assignee: ISTITUTO DI RICERCHE DI BIOLOGIA MOLECOLARE P. AN, Pomezia (IT)

(21) Appl. No.: 12/084,121

(22) PCT Filed: Oct. 26, 2006
(86) PCT No.: PCT/EP2006/067835

§ 371 Date: Jun. 8, 2009

(30) Foreign Application Priority Data
Oct. 28, 2005 (GB) 0522008.2

(51) Int. Cl. C12Q 1/25; C12N 9/00; C12N 5/10

(52) U.S. Cl. 435/4; 435/183; 435/325

(57) ABSTRACT

The present invention relates to an assay specific for histone deacetylases HDAC1 and/or 2 inhibitors which comprises: (i) incubating an HDAC1 and/or 2 enzymes(s) together with a protein that contains the SANT and ELM2 regions, found in MTA proteins such as MTA-2, MTA-1, MTA-3 and also found in CoREST, CoREST2, CoREST3 and MI-ER1, in a suitable assay buffer (ii) adding the potential HDAC inhibitor and a suitable substrate and incubating (iii) stopping the incubation and determining the effect the putative HDAC inhibitor has had on enzyme activity by comparison with standards.

The present invention relates to a high efficiency assay, specific for histone deacetylases 1/2 (HDAC1/2) that is suitable for the screening of HDAC subtype-selective inhibitors.

Reversible histone acetylation has been identified as a major regulator of eukaryotic gene transcription. Lysine residues in histone tails are acetylated by histone acetyltransferases (HATs) that function as transcriptional coactivators. The acetylation of histones results in a less restrictive chromatin structure than is generally associated with transcriptional activation. Histone deacetylases (HDACs) reverse the reaction catalyzed by HATs, leading to a repressive chromatin structure. Multiple HDACs of three major classes have been identified. Class I HDACs include HDACs 1, 2, 3 and 8 and are homologous to yeast Rpd3 deacetylase, whereas class II deacetylases including HDACs 4, 5, 6, 7, and 9 are more similar to yeast Hda1. HDAC11 exhibits both similarities and distinctive features as compared with all other subtypes. A third class of HDACs includes homologs of the yeast Sir2 silencing protein which requires a NAD cofactor for activity. HDACs have been found to function in vivo as large multiprotein complexes that are targeted by DNA binding proteins to actively repress gene transcription.

HDAC3 normally exists in tight and stoichiometric complexes with corepressors N-CoR and SMRT. The enzyme activity of HDAC3 requires SMRT or N-CoR, which interact with and activate the deacetylase via a DAD domain (deacetylase activating domain), that includes one of two SANT motifs present in both corepressors. DAD comprises an N-terminal DAD-specific motif and a C-terminal SANT-like domain.

Numerous proteins involved in transcription contain a conserved SANT motif, whose function is not well understood (Boyer, L. A., Latek, R. R. and Peterson, C. L. (2004) Nat. Rev. Mol. Cell. Biol., 5: 158-163). Recombinant HDAC3 is inactive and activation occurs upon coexpression of the deacetylase and the cofactor SMRT/N-CoR in cells (like HeLa cells) or by in vitro transcription-translation of HDAC3 and SMRT/N-CoR with Rabbit Reticulocyte Lysate (RRL).

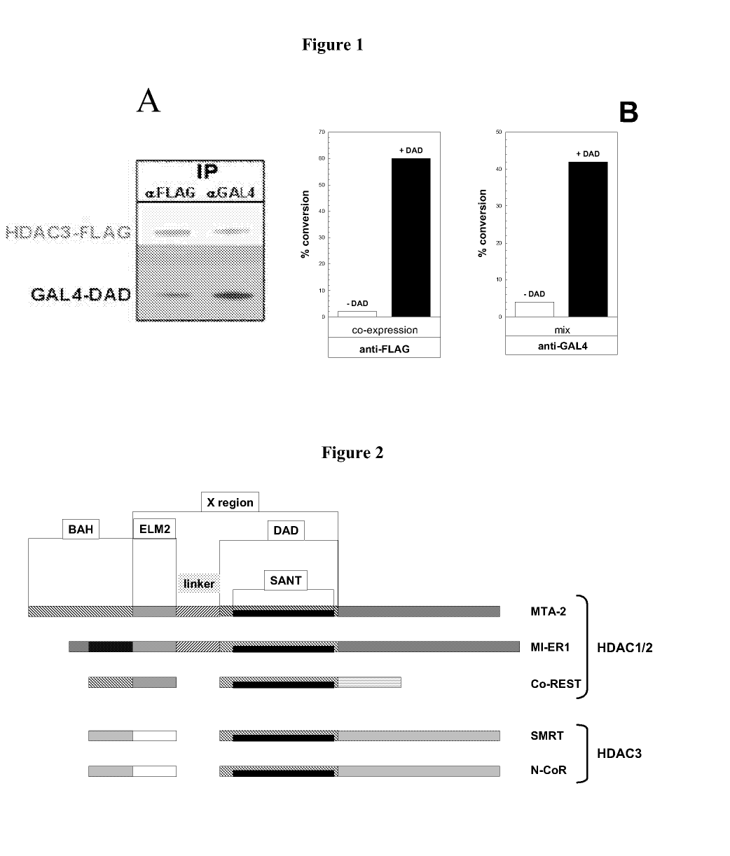

To confirm published evidence, a C-terminally flagged HDAC3 (HDAC3-FLAG) was cotransfected in cells with a construct containing the DAD region of N-CoR [(DAD was fused to the C-terminus of the DNA Binding Domain of GAL4 (GAL4-DBD)]. Additionally, HDAC3-FLAG and GAL4-DAD were individually expressed and mixed. The complexes were immunoprecipitated with anti-FLAG (for HDAC3) or anti-GAL4 (for DAD) (FIG. 1A), and incubated with a histone H4-derived octamer peptide substrate containing a single acetylated lysine. The activity is indicated as % conversion of the acetylated substrate (FIG. 1B) and confirms that SMRT/N-CoR DAD is essential for the interaction and activation of HDAC3.

HDAC1 and HDAC2 share 84% identity and it is unclear whether they have distinct functions, but they appear to play complementary roles in transcriptional repression. Known HDAC1/2 complexes contain transcriptional corepressors mSin3A (Hassig, C. A., Fleischer, T. C., Billin, A. N., Schreiber, S. L. and Ayer, D. E. (1997) Cell, 89: 341-348), CoREST (Corepressor of REST; You, A., Tong, J. K., Grozinger, C. M. and Schreiber, S. L. (2001) Proc. Natl. Acad. Sci. U.S.A., 98: 1454-1458), MTA-2 (Metastasis-associated protein 2 in NuRD complex; Zhang, Y., Ng, H.-H., Erdjument-Bromage, H., Tempst, P., Bird, A. and Reinberg, D. (1999) Genes Dev., 13: 1924-1935), MI-ER1 (Mesoderm induction early response 1; Ding, Z., Gillespie, L. L. and Paterno, G. D. (2003) Mol. Cell Biol., 23: 250-258).

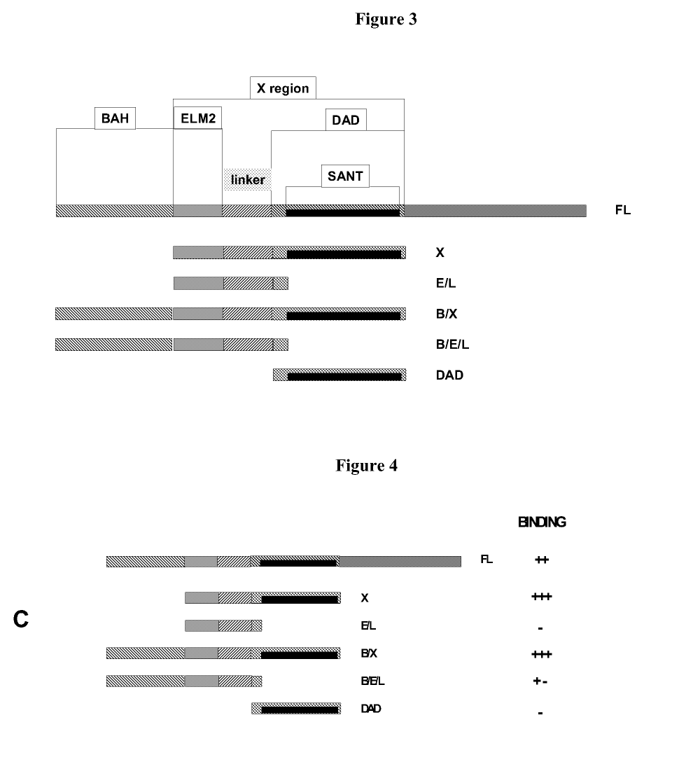

A SANT motif is present in CoREST, MTA-2 and MI-ER1 (FIG. 2). We found that in all these proteins the homology is not limited to SANT, but continues at the N-terminus, permitting the definition of a DAD-homology region, as in SMRT/N-CoR (FIG. 2). HDAC1/2 corepressors have in common another domain, ELM2, first described in Eg1-27, a Caenorhabditis elegans protein that plays a fundamental role in patterning during embryonic development (Solari, F., Bateman, A. and Ahringer, J. (1999) Development, 126: 2483-2494). That part of the protein that encompasses both ELM2 and DAD, is designated the X-region in FIG. 2 (hereinafter referred to as the “X-region”). MTA-2 corepressor has an additional N-terminal domain, BAH (Bromo-adjacent homology); Goodwin, G. H. and Nicolas, R. H. (2001) Gene, 268:1-7; FIG. 3), that has been identified in a number of proteins involved in gene transcription and repression and is likely to be involved in protein-protein interactions. Based on amino acid sequence homology a number of additional proteins containing a X-region can be identified, for example MTA-1, MTA-3, CoREST, CoREST2 and CoREST3.

The SANT domain of MTA-2 seems to contribute to the binding to HDAC1 (Humphrey, G. W., Wang, Y., Russanova, V. R., Hirai, T., Qin, J., Nakatani, Y. and Howard, B. H. (2001) J. Biol. Chem., 276: 6817-6824), and in CoREST looks essential for HDAC1 functional interaction (You, A., Tong, J. K., Grozinger, C. M. and Schreiber, S. L. (2001) Proc. Natl. Acad. Sci. U.S.A., 98: 1454-1458). On the other hand, in MI-ER1 Ding et al. mapped the domain responsible for recruiting HDAC1 activity and mediating transcriptional repression to a region containing ELM2; their analysis shows that the SANT domain of MI-ER1 is dispensable for productive interaction with HDAC1.

A number of MTA-2 deletion mutants were constructed and fused to GAL4-DBD C-terminus (FIG. 3 and experimental). After coexpression with flagged-HDAC1 and co-immune purification with anti-FLAG, the association of the MTA2 mutants with HDAC1 was measured (FIG. 4).

As shown in FIG. 4, neither the DAD-homology region nor the ELM2 domain were sufficient for the binding of the corepressor to HDAC1. The complete X-region, comprising both domains, was necessary and sufficient for the interaction in these experiments, whereas BAH appeared to be dispensable.

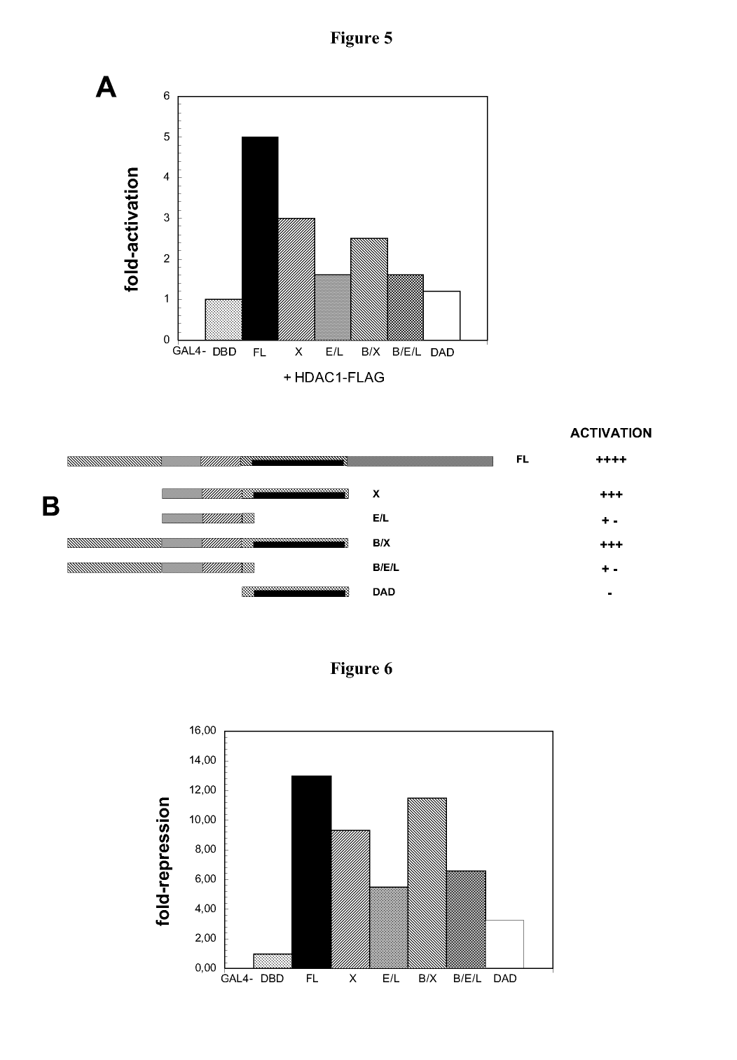

The same coimmunopurified complexes were incubated with the H4-derived acetylated peptide substrate. All samples were normalised to contain HDAC1-FLAG at the same low, sub-optimal concentration (0.25 nM) (FIG. 5A). At this concentration, HDAC1 was not active in the absence of the recombinant corepressors: to observe enzymatic activity, it was necessary to use at least 5 nM of HDAC1-FLAG (not shown).

The data clearly show that the full-length corepressor MTA-2 gave the best activation of HDAC1: a 5-fold-activation with respect to the background level (DBD) was measured as an average of three independent experiments (FIG. 5A). The activation capacity of the constructs is summarized in FIG. 5B.

Confirmatory results were obtained in a transcriptional repression assay, in which GAL4-MTA2 expression vectors were cotransfected into cells together with a GAL4-dependent reporter construct (FIG. 6).

The repression was HDAC-dependent, since in the presence of an HDAC inhibitor transcription was restored (data not shown).

From these experiments, it can be concluded that:
Removal of the SANT domain from MTA-2 based chimeras decreases the binding, the activation potential and the transcription repressive function;
The ELM2 domain is essential, but is not sufficient for HDAC1 function;
The minimal activation domain for HDAC1 is the X-region, that encompasses both ELM2 and DAD-homology region;
Maximal activation of HDAC1 is obtained with the full length MTA-2 corepressor.

Similar results were obtained repeating (some of) the experiments with HDAC2 and with another corepressor, CoREST.

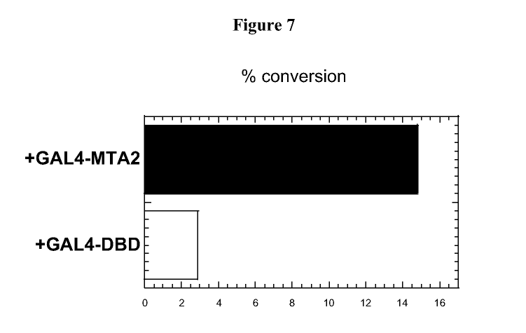

The ability of the MTA-2 corepressor, or fragments of it containing the X-region, to enhance HDAC1 and/or 2 catalytic activities represents a novel finding. In particular, by co-purifying HDAC1 and 2 with the complete MTA-2 corepressor, it was possible to enhance the catalytic efficiency of the deacetylase in the order of one log (FIG. 7 and not shown). To observe enzymatic activity with HDAC1/2, it is necessary to use at least 5 nM of the deacetylase, whereas in the presence of the corepressor a concentration of 0.25 nM is sufficient). This finding, which has not previously been reported, enables the establishment of a high efficiency assay specific for histone deacetylase 1 and 2 that is suitable for screening of HDAC subtype-selective inhibitors since sub-nanomolar enzyme concentrations are sufficient to obtain suitable substrate conversions.

Histone deacetylases are known to play key roles in the regulation of cell proliferation, consequently inhibition of HDACs has become an interesting approach for anti-cancer therapy. To this purpose, it is very important to devise subtype-selective enzymatic assays with small amounts of enzymes.

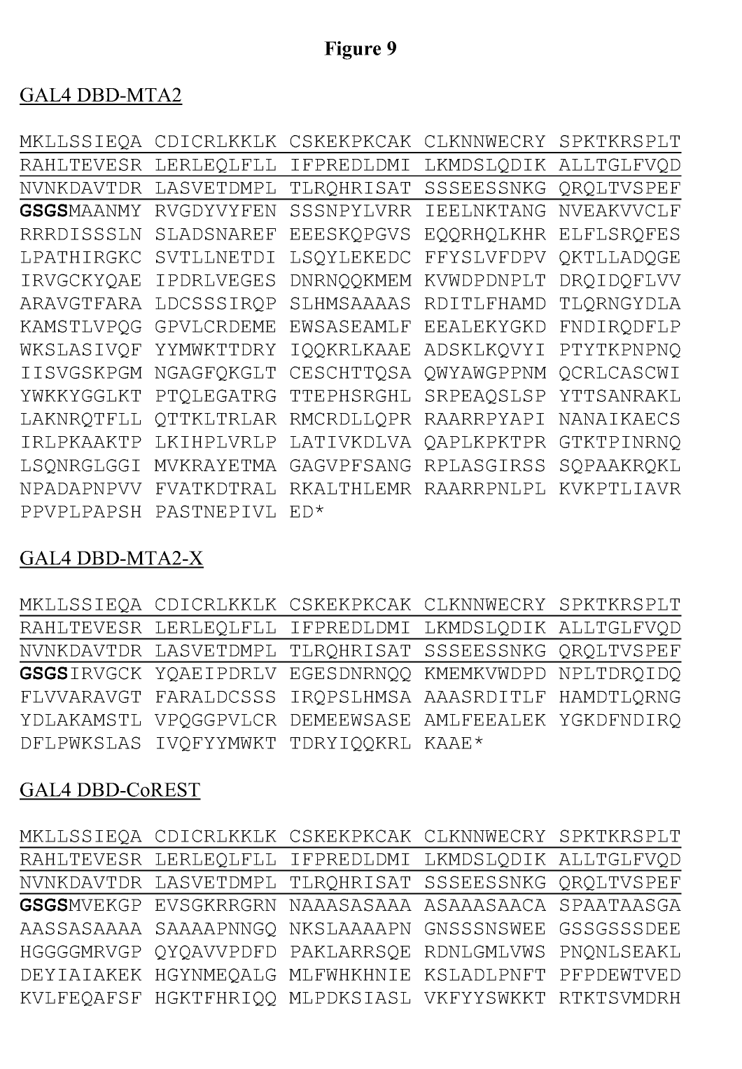

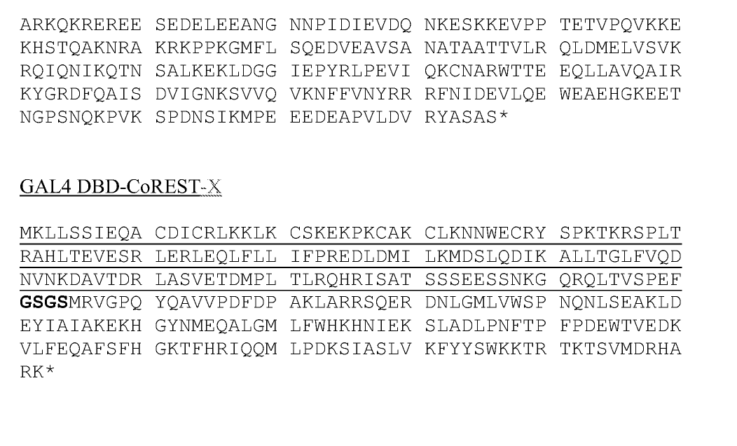

FIG. 9 shows GAL DBD, underlined, and the GSGS linker which is in bold.

Accordingly, in a first aspect the present invention provides an assay specific for histone deacetylases HDAC1 and/or 2 inhibitors which comprises:
(i) incubating an HDAC1 and/or 2 enzyme(s) together with a protein that contains the SANT and ELM2 regions, found in MTA proteins such as MTA-2, MTA-1, MTA-3 and also found in CoREST, CoREST2, CoREST3 and MI-ER1, in a suitable assay buffer
(ii) adding the potential HDAC inhibitor and a suitable substrate and incubating
(iii) stopping the incubation and determining the effect the putative HDAC inhibitor has had on enzyme activity by comparison with standards.

Suitably the protein in step (i) is a recombinant protein that is preferably expressed in mammalian cells. The protein suitably contains full-length MTA-1, MTA-2, MTA-3, CoREST, CoREST2, CoREST3, MI-ER1 or a protein that has substantial sequence identity with one of these proteins (and suitably at least 80% and preferably at least 85% sequence identity) and contains the X-region (as herein described) or a fragment of one of these proteins containing the X-region (as herein described). Preferably, the protein is full-length MTA-2.

In a further embodiment, the protein in step (i) is a recombinant protein that is preferably expressed in mammalian cells. The protein suitably contains an X-region that has substantial sequence identity with the X-region of an MTA protein, such as MTA-2, MTA-1 or MTA-3, or the X-region of CoREST, CoREST2, CoREST3 or MI-ER1 (and suitably at least 80% and preferably at least 85% sequence identity).

The present invention further relates to a reconstituted enzymatic complex containing HDAC1 or HDAC2 and a protein that consists essentially of full-length MTA-2 or a fragment of this that contains its X region.

Recombinant cells that have been transformed with heterologous DNA to express HDAC1 and/or HDAC2 and at least the amino acids of the X region are also provided by the present invention.

In particular aspects of performing the method, aliquots of the enzyme, and in particular the recombinant enzyme, are provided together with the protein containing the SANT and ELM2 motifs. Each aliquot is deposited in the well of a microplate. Serial dilutions of a test analyte being tested for HDAC inhibitor activity are made and each dilution is added to a separate well of the microtitre plate containing the cells. Optionally, the method can include serial dilutions of a known HDAC inhibitor as a positive control and further can include negative controls. Each aliquot is deposited in the well of a microplate. A plurality of test analytes being tested for HDAC inhibitor activity are each added to a separate well of the microtiter plate containing the enzyme together with the protein containing the SANT and ELM2 motifs. In further aspects, serial dilutions of the plurality of analytes are made and each dilution is added to a separate well of the microtiter plate containing the cells.

The following examples serve to illustrate the present invention and the manner in which it can be performed.

### EXAMPLES

### Flagged-HDAC1/2 Co-Expression With GAL4-MTA2/CoREST in HeLa Cells: Transient Transfection, Preparation of Soluble Extracts, and Affinity Purification

The mammalian expression vectors used for transient co-transfection into HeLa cells were the following: pCDNA HDAC1-FLAG or pCMV HDAC2-FLAG together with pCDNA GAL4 DBD-MTA2/CoREST, or the corresponding deletion mutants.

10⁷ HeLa cells were transfected with 7.5 micrograms of pCDNA-HDAC1-FLAG or pCMV-HDAC2-FLAG together with 7.5 micrograms of pCDNA-GAL4DBD-MTA2/CoREST plasmid DNAs using Lipofectamine reagent (Invitrogen) according to the manufacturer recommendations.

Cells were collected in 1×PBS 24 hr after transfection, centrifuged at 1500×g for 5 min at 4° C., and washed twice with 1×PBS. After centrifugation, cell pellets were stored frozen at −80° C. Cell lysates were obtained by resuspending the cell pellet in 1 ml of hypotonic lysis buffer (20 mM Hepes pH 7.9, 0.25 mM EDTA, 10% glycerol, 1 mM PMSF, Complete EDTA-free protease inhibitors cocktail from Boehringer), followed by incubation 15′ on ice, cell disruption in a 2 ml Douncer (B, 25 strokes), and addition to the homogenate of 150 mM KCl and 0.5% NP40 (isotonic lysis buffer: ILB). Soluble whole cell extracts were obtained by sonicating samples for 30 sec twice (output 5/6, duty cycle 90, timer constant), followed by a 1-hr incubation on a rotating wheel at 4° C. Upon centrifugation at 12000 rpm in SS34 rotor for 30 min at 4° C., the clear supernatant (soluble protein extract) was collected, and the total protein concentration determined using the BioRad reagent. Flagged-HDAC concentration in the extract was estimated by running 4, 8, and 16 micrograms of total protein on a 4-12% SDS-PAGE minigel together with 8-16 ng of reference protein, followed by Western blot analysis with an anti-FLAG alkaline phosphatase-conjugated monoclonal antibody (M2-AP, A9469, SIGMA). The expression levels of GAL4 DBD-MTA2/CoREST chimeric protein in the same extract was assessed on the same immunoblot developed with an anti-GAL4 rabbit polyclonal antibody (Santa Cruz Biotechnology, sc-577).

Soluble protein extracts containing flagged HDAC1 or HDAC2 in combination with each of the GAL4 DBD-MTA2 or CoREST variants were applied on anti-FLAG M2 Affinity gel (A2220, SIGMA) prepared according to the manufacturer recommendations and equilibrated in ILB. Gel matrix and soluble protein extract were mixed (10 microliters of gel matrix for each 2 micrograms of flagged-HDAC), and incubated O.N. on a rotating wheel at 4° C. The gel matrix was recovered by centrifugation, and washed once with ILB, twice with ILB containing 0.1% NP-40, and 2 more times in elution buffer [50 mM Hepes pH 7.4, 5% glycerol, 0.01% Triton X-100, 100 mM KCl. HDAC complexes were eluted by adding to the gel matrix 10 volumes of elution buffer containing 100 micrograms/ml of 3×FLAG peptide (F4799, SIGMA), followed by a 1 hr-incubation on a rotating wheel at RT. Upon centrifugation, eluted proteins were collected in the supernatant and flagged-HDAC1/2 concentration estimated by anti-FLAG Western blot analysis using 10, 20, and 30 ng of reference protein for quantification. The presence of each of the HDAC-associated GAL4 DBD-MTA2/CoREST variants was assessed on the same immunoblot developed with anti-GAL4 antibody.

Flagged-HDAC1/2 and GAL4-MTA2/CoREST Expression by Coupled in vitro Transcription-Translation and Affinity Purification

The TNT Quick Coupled Transcription/Translation System (Promega) was used following the manufacturer recommendations. For mixing experiments, 4 micrograms of each of the expression plasmid DNAs were used to drive the synthesis in vitro of the corresponding protein (flagged-HDAC or GAL4 DBD-MTA2/CoREST) in a reaction volume of 120 microlitres. For coexpression experiments, 0.8 micrograms of flagged-HDAC1/2 expression plasmid DNA and 6 micrograms of each cofactor expression plasmid DNA were added to the same reaction in a volume of 200 microlitres. After completion of the protein synthesis step, for mixing experiments, 25 microlitres of the HDAC1/2 TNT reaction were mixed with 100 microlitres of the cofactor TNT reaction and the final volume was brought to 200 microlitres by adding 75 microlitres of a mock TNT reaction. Samples were then incubated at room temperature for 10-15 minutes. 200 microlitres of HDAC1/2 +cofactor mixtures were applied on 30 microlitres anti-FLAG M2 Affinity gel prepared according to the manufacturer recommendations and equilibrated in M2-IP buffer (20 mM Hepes pH 7.9, 300 mM KCl, 0.25 mM EDTA, 10% glycerol, 0.1% Tween 20, COMPLETE EDTA-free protease inhibitors cocktail). After an incubation O.N. on a rotating wheel at 4° C., the gel matrix was recovered by centrifugation, and washed once with M2-IP buffer, twice with M2-IP buffer containing 150 mM KCl, and 2 more times in HDAC HPLC activity buffer (50 mM Hepes pH 7.4, 5% Glycerol, 0.01% Triton X100, 0.1 mg/ml BSA). One fifth of the sample was analyzed by SDS-PAGE and Western blot. The remaining gel matrix was recovered by centrifugation and incubated in a final volume of 100 microliters of HDAC activity buffer for HPLC deacetylase assays.

### HDAC HPLC Activity Assay

2 microliters of the test compound solution in 100 % DMSO (50×) were added to 100 microliters of assay buffer (50 mM Hepes pH 7.4, 5% Glycerol, 0.01% Triton X100, 0.1 mg/ml BSA) containing a fixed concentration of recombinant enzyme. After pre-incubation at room temperature for 15 min, 2 microliters of a 200 μM pre-dilution in DMSO of the fluorescent histone H4 (AcK 16) peptide substrate (final concentration: 4 μM) were added [peptide substrate: Mca-GAK(ε-Ac)RHRKV-NH₂ λex 325 nM; λem 393 nM]. The deacetylase reaction was performed at room temperature for 4 hours and stopped by addition of 20 μl of 10% Trifluoro Acetic Acid (TFA) aqueous solution. The percentage of peptide substrate converted to the corresponding deacetylated product was analyzed by HPLC with a Merck-Hitachi chromatograph on a Beckman (4.6 mm×5 cm) C18 column eluted at 1 ml/min with a 24 ml linear gradient from 5% to 40% acetonitrile in H₂O, followed by a steep gradient (1 ml) up to 95% acetonitrile and an additional 3ml-step at 95% acetonitrile.

Most exogenously expressed recombinant HDACs were found to be inactive or scarcely active. On the contrary, recombinant enzymes expressed in mammalian cells have the same propensity as their endogenous counterparts to interact with specific regulatory polypeptides that affect their activity. The main hurdle to using mammalian cells for large-scale HDAC production resides however in the relatively low protein yields. To observe enzymatic activity with HDAC1/2, it is necessary to use at least 5 nM of the deacetylase. We found that HDAC1/2 activity is enhanced in the order of one log in the presence of the corepressor MTA-2. This allows scaling-up of the assay throughput, since sub-nanomolar enzyme concentrations (0.25 nM) are sufficient to obtain suitable substrate conversions.

### Amino Acid Sequence Alignment of Proteins Containing the X-Region

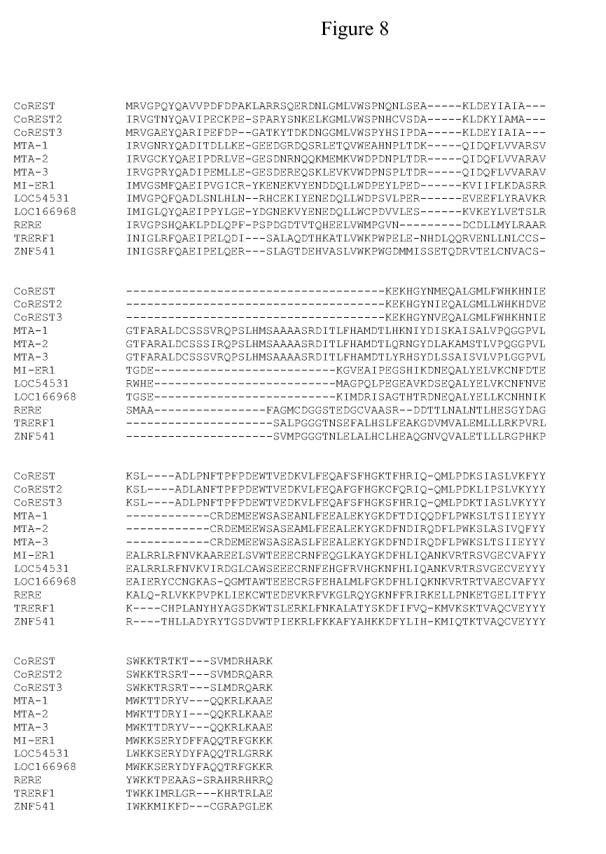

Additional proteins containing the X region were identified by searching protein sequence databases using the amino acid sequence of the X-region from MTA-2. CoREST or MI-ER1 as query. The results are shown in FIG. 8.

1. An assay for identifying a potential inhibitor of one of an HDAC1 or HDAC 2 enzyme activity comprising the steps of:
(i) incubating one of said HDAC1 or HDAC2 enzyme with a protein that contains the SANT and ELM2 regions, found in MTA proteins such as MTA-2, MTA-1, MTA-3 and also found in CoREST, CoREST2, CoREST3 and MI-ER1, in a suitable assay buffer;
(ii) adding the potential inhibitor and a suitable substrate under conditions suitable for enzymatic activity to occur; and
(iii) determining the effect of the putative HDAC inhibitor on enzyme activity relative to a standard.

2. The assay according to claim 1; wherein the protein in step (i) comprises a full-length protein selected from the group consisting of MTA-1, MTA-2, MTA-3, CoREST, CoREST2, CoREST3, and MI-ER1 or a protein that has at least 80% sequence identity with one of said proteins selected from the group consisting of MTA-1, MTA-2, MTA-3, CoREST, CoREST2, CoREST3, and MI-ER1 and comprises an ELM2 and DAD regions.

3. The assay according to claim 2 wherein the protein in step (i) is full-length MTA-2.

4. A reconstituted enzymatic complex comprising HDAC1 or HDAC2 and full-length MTA-2 or a fragment thereof comprising the ELM2 and DAD regions.

5. A recombinant cell transformed with heterologous a nucleic acid molecule encoding a histone deacetylase selected from the group consisting of HDAC1 and HDAC2 and a protein comprising the ELM2 and DAD regions.
